(19) United States
(12) Patent Application Publication
(10) Pub. No.: US 2025/0295507 A1
(43) Pub. Date: Sep. 25, 2025

(54) CATHETER FOR DELIVERING A NASAL STENT

(71) Applicant: Intersect ENT, Inc., Menlo Park, CA (US)

(72) Inventors: Yiwen Tang, Sunnyvale, CA (US); Rahul Bitla, Sammamish, WA (US); Alex Christopher Drake, Santa Cruz, CA (US); Chang Lee, Menlo Park, CA (US)

(21) Appl. No.: 19/086,430

(22) Filed: Mar. 21, 2025

Related U.S. Application Data
(60) Provisional application No. 63/568,460, filed on Mar. 22, 2024.

(51) Int. Cl. A61F 2/915; A61F 2/958; A61M 25/10

(52) U.S. Cl. CPC A61F 2/915; A61F 2/958; A61M 25/1011; A61F 2230/0056; A61F 2230/0069; A61F 2240/00; A61F 2250/0067

(57) ABSTRACT

A nasal stent includes an elongated tubular body having proximal and distal ends extending along a longitudinal axis. The elongated body includes a repeating pattern of W-shaped members extending around the tubular body separated by longitudinally-oriented and offset struts. The elongated tubular body is selectively transitionable between an expanded configuration and a crimped configuration, wherein the pattern of W-shaped members and struts interleave allowing the elongated tubular body to maintain substantially the same length when disposed in the expanded configuration and the crimped configuration.

### CROSS-REFERENCE TO RELATED APPLICATIONS

This application claims the benefit of and priority to U.S. Provisional Application No. 63/568,460 filed on Mar. 22, 2024, the entire contents of which is being incorporated herein.

### TECHNICAL FIELD

The present disclosure relates to devices and delivery systems for the treatment of sinus and nasal conditions. More particularly, the present disclosure relates to a drug-eluting, implantable stent and delivery system for accessing, positioning and implanting the stent into a sinus or nasal cavity for relieving chronic sinusitis and/or other inflammatory sinus conditions.

### BACKGROUND

Sinusitis is a condition characterized by inflammation of the mucosal lining of the sinuses. Chronic Rhinosinusitus (CRS) is defined as swelling and inflammation of the sinuses, interfering with the way mucus normally drains and may affect as many as 5-10% of the adult world population. Current therapies include corticosteroid treatment to reduce inflammation, and surgical treatment to remove inflamed tissue. After surgery, steroid treatment may be needed to reduce post-surgical inflammation but is often not effective. Topical applications such as sprays, creams or gels is also inconsistent due to factors such poor absorption in the affected sinus due to obstructions or limited contact time with the sinus mucosa resulting in low absorption of the steroid.

Implantable drug-eluting stents (combination of stent disposed atop a delivery system) were introduced several years ago to solve many of the aforementioned issues with simply administering steroids, namely, the drug-eluting stent: solves the issue of delivery, location and contact time as the surgeon is able to precisely deliver the drug-eluting stent to the infected or inflamed area for treatment; maintains the inflamed passageway open by virtue of its radial expansion strength; and releases controlled amounts of medicament over a prolonged period of time. The stents may be either or both biodegradable over a pre-set period of time or removeable over a pre-set period of time.

The wide success of the drug-eluting stents for CRS and other sinus conditions led to the desire for better drug-eluting stents with higher drug-eluting capacity, longer release periods and greater radial expansion strengths. Moreover, a need was developed to deliver the new higher capacity stents to deeper locations, new locations and into more difficult-to-access, sinus locations requiring articulatable delivering systems, bendable stents (or stents made from a bendable material) and/or stents with delivery systems with enhanced access features.

### SUMMARY OF THE INVENTION

Provided in accordance with the present disclosure is a nasal stent which includes an elongated tubular body having proximal and distal ends extending along a longitudinal axis. The elongated body includes a repeating pattern of W-shaped members extending around the tubular body separated by longitudinally-oriented and offset struts. The elongated tubular body is selectively transitionable between an expanded configuration and a crimped configuration, wherein the pattern of W-shaped members and struts interleave allowing the elongated tubular body to maintain substantially the same length when disposed in the expanded configuration and the crimped configuration.

Provided in accordance with another embodiment in accordance with the present disclosure is a catheter for delivering a nasal stent which includes a housing having an elongated shaft extending distally therefrom. The catheter also includes a pair of inflation ports disposed on the housing, each inflation port communicating with a lumen defined through the elongated shaft. An access balloon is disposed proximate a distal end of the shaft, the access balloon communicating with a first inflation port of the pair of inflation ports. A delivery balloon is disposed proximal to the access balloon and communicates with a second inflation port of the pair of inflation ports, the delivery balloon, when selectively inflated, expanding the delivery balloon. A nasal stent is crimped atop the delivery balloon, the nasal stent being made from a material allowing the nasal stent to: expand against the inner peripheral tissue walls of a nasal cavity upon inflation of the delivery balloon; maintain radial strength after expansion; and elute a medicament or therapeutic agent to the inner peripheral tissue walls of the nasal cavity. A sleeve houses the nasal stent atop the delivery balloon during navigation to the surgical site. A balloon actuator is configured to selectively and independently regulate the inflation of each balloon and a nasal stent delivery actuator is configured to regulate the exposure of the nasal stent from the sleeve.

Provided in accordance with the present disclosure is a method of delivering a nasal stent which includes engaging a distal end of an elongated shaft of a catheter with an entry of a nasal passage and inflating an access balloon via a first inflation port of a pair of inflation ports disposed on a housing of the catheter to bluntly dissect obstructions as the elongated shaft is navigated through the nasal passage to an infected area. The method further includes positioning a delivery balloon disposed atop the elongated shaft in the infected area, the delivery balloon configured to support a nasal stent thereon in a crimped configuration and inflating the delivery balloon via a second inflation port of a pair of inflation ports disposed on the housing to radially expand the nasal stent against the inner peripheral tissue walls of the nasal passage in the infected area, the nasal stent being made from a material allowing the nasal stent to: expand against the inner peripheral tissue walls of a nasal passage upon inflation of the delivery balloon; maintain radial strength after expansion; and elute a medicament or therapeutic agent to the inner peripheral tissue walls of the nasal passage to treat the infected area.

### BRIEF DESCRIPTION OF THE DRAWINGS

FIG. 1A is an enlarged view of a nasal stent in an expanded configuration disposed atop a shaft of a delivery catheter;

FIG. 1B is a greatly-enlarged view of the nasal stent of FIG. 1A highlighting the W-shaped pattern design of the stent in accordance with the present disclosure;

FIG. 2A is an enlarged view of the nasal stent of FIG. 1A shown crimped atop a delivery balloon forming part of the delivery catheter;

FIG. 2B is a greatly-enlarged view of the nasal stent of FIG. 2A shown crimped atop the delivery balloon;

FIG. 3 is a top view of the nasal stent in an expanded configuration disposed atop the shaft of the delivery catheter showing the mechanical integrity of the stent during angular manipulation of the shaft;

FIG. 4 is a side view of an alternate embodiment of delivery catheter according to the present disclosure having dual inflation ports for inflating two balloons;

FIG. 5A is a greatly-enlarged, transparent, side view of the stent shown crimped atop a delivery balloon for use with the catheter of FIG. 4;

FIG. 5B is a greatly-enlarged, side view of the two balloons for use with the catheter of FIG. 4; one balloon utilized for accessing the nasal passage and one balloon used for delivering the stent;

FIG. 5C is a side view of the access balloon being inflated for bluntly dissecting and removing obstacles while navigating to the surgical site and for clearing the surgical site for delivery of the stent; and

FIG. 5D is a side view of the delivery balloon being inflated for positioning delivering the stent to the surgical site.

Like reference symbols in the various figures of the drawing indicate like elements. The elements in the drawing are not to scale.

### DETAILED DESCRIPTION

The following detailed description discusses certain embodiments and is not to be taken in a limiting sense. All weights, amounts and ratios herein are by weight, unless otherwise specifically noted.

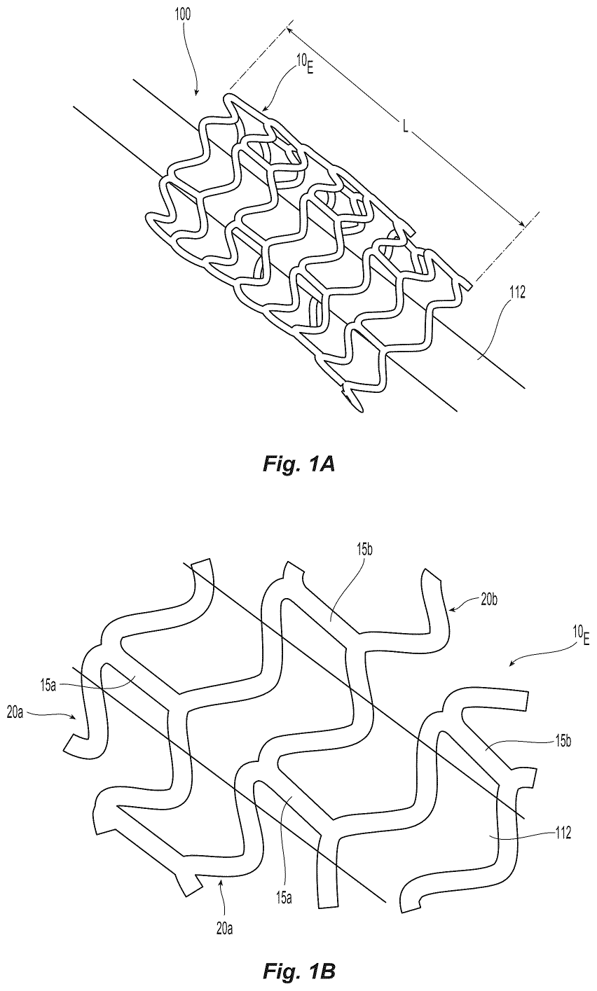

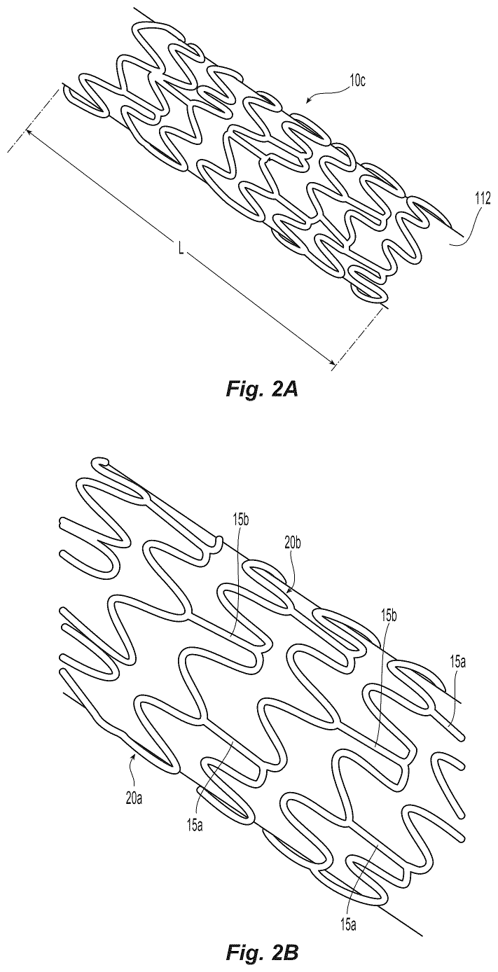

Referring initially to FIGS. 1A and 1B, a drug-eluting stent 10 is shown in an expanded state atop a shaft 112 of a delivery device, e.g., balloon catheter 100. Stent 10 may be formed by laser cutting, stamping, die-cutting or any other way known in the art and may be made from a variety of different materials depending upon a particular purpose. For example, stent 10 may be made out of Poly(lactic-co-glycolic) Acid (PLGA): PLGA, Polycaprolactone (PCL): PCL or Poly(lactic) Acid (PLA): PLA, formed into tubing (See FIG. 1A), and then laser cut into a desired design shape. The tube-like stent 10 is then crimped onto the catheter 100 with a crimping tool (not shown).

As best shown in FIG. 1B, stent 10 includes a series of repeating, W-shaped members, e.g., W-shape member 20a, each separated by a respective longitudinal strut, e.g., strut 15a, arranged in an off-set, repeating pattern relative to one another resulting in a W-shaped pattern, i.e., W-shaped members along the stent 10 forming an interleaved lattice of W-shaped members with off-set longitudinal struts 15a, 15b. Forming the stent 10 with the interleaved lattice design of the W-shaped members 20a and 20b together with the offset longitudinal struts 15a and 15b enables (hereinafter “the W-shaped pattern”) the stent 10 to be crimped by the crimping tool from a first, expanded configuration (See stent 10E of FIGS. 1A and 1B) to a second crimped configuration atop the delivery device, e.g., balloon catheter 100 (See stent 10c of FIGS. 2A and 2B). Moreover, the W-shaped pattern enables the stent 10 to be crimped by the crimping tool from the first, expanded configuration to a second crimped configuration atop the balloon catheter 100 with very little, if any, effect on the overall length “L” of the stent 10.

The W-shaped pattern allows the stent 10 to be crimped atop a deflated balloon 50 in a significantly reduced radial profile (from about 7.5 mm OD to about 3.0 mm OD) facilitating delivery (FIG. 2B). As a result, a doctor is able to utilize a small, drug-coated stent 10 in an in-patient or in-office setting eliminating the need for a hospital visit. Further, the W-shaped patterns allows the stent 10 to be repeatedly expanded (and sometimes bent) while the shaft 112 is bent during delivery (up to about 80 degrees) without affecting the mechanical integrity, radial strength, or polymer properties of the stent 10 when eventually deployed.

As mentioned above, the stent 10 is radially expandable and is also radially compressible or radially resilient, in that the stent 10 can repeatedly be converted from the stent's 10 expanded state to the stent's 10 unexpanded state and back by radially compressing and relaxing the stent 10. When in an expanded state, stent 10 is sized and shaped for introduction and operable engagement with a sinus, nasal cavity or passage without inhibiting the free passage of air, exhalation gases, mucus or other fluids into and out of the sinus, nasal cavity or passage.

Stent 10 is typically sized to just fit within such cavity or passage when uninstalled and unrestrained, or so as to be slightly compressed when installed in and restrained by such cavity or passage. Prior to installation, stent 10 is radially compressed or crimped to a diameter suitable for loading atop the delivery device, e.g., balloon catheter 100 (or other known delivery device) and inserted into such cavity or passage. When compressed, typical stents have a smaller diameter and greater axial length than when expanded and unconstrained. Stent 10 has a smaller diameter but maintains virtually (with about 5%) the same length “L” in both the compressed and expanded states. As mentioned above, stent 10 has an expanded outside diameter of about 7.5 mm to and a compressed outside diameter of about 3.0 mm when disposed atop shaft 112.

Exemplary stents are sold by Intersect ENT, Inc., e.g., the PROPEL™ stent, the SINUVA™ stent and the PROPEL Mini stent. Both stents are biodegradable radially expandable polymeric stents loaded with mometasone furoate, and both are designed to be delivered in compressed form into the ethmoid sinus as is or following functional endoscopic sinus surgery (“FESS”), whereupon they expand, slowly degrade, elute mometasone furoate to nearby tissues, and can help treat conditions such as chronic rhinosinusitis. The original version of the PROPEL™ stent has a compressed diameter of 5.2 mm for installation and about a 60 mm diameter when unconstrained. The “PROPEL Mini™” version is used in patients with less extensive surgery or smaller anatomy, and has a compressed diameter of 4 mm for installation and about a 40 mm diameter when unconstrained.

As will be discussed in more detail below, stent 10 and the other embodiments disclosed herein may be drug-eluting or non-drug-eluting and may be fully biodegradable, partially biodegradable or non-biodegradable. The outer surface of the stent 10 may be configured to enhance retention of the stent 10 against the internal tissue surface of the sinus or nasal passages. Alternatively, the radial force of the stent 10 may be sufficient to maintain the stent 10 in a desired location within the nasal passage. Further, it is envisioned that one or more of the features of the interleaved lattice design of the W-shaped pattern, namely, the W-shaped members 20a, 20b and or the offset longitudinal struts 15a, 15b, may interact with the internal tissue of the nasal passage and, together with the radial force of the stent 10, work to embed themselves when the stent 10 expands into position.

The performance characteristics of stent 10 are far superior to other known stents for treating sinus and nasal conditions. The combination of the material making up the stent 10, the design of the W-shaped pattern of the stent 10, the precision of cutting the tube-shaped lattice of the W-shaped members 20a, 20b making up the W-shaped pattern, and the method for curing the stent 10 to increase the radial strength are all contributing factors playing a part and which, together, enhance the performance of stent 10.

Briefly, the process for making the stent 10 starts with extruding plastic into a tubular form via a heat expansion and extrusion process. The following polymer resins are particularly well-suited for use as the stent 10: a mixture of Purasorb PLG1017 and Purasorb PLG8531; Purasorb PLG8531; Purasorb PL32; and Purasorb PLC9515. Purasorb PLG1017 is a copolymer with 10% L-lactide and 90% glycolide with resin IV 1.7 dl/g; Purasorb PLG8531 is a copolymer of 85% L-lactide and 15% glycolide with resin IV 3.1 dl/g; Purasorb PL32 is a poly (L-lactide), resin IV 3.2 dl/g; and Purasorb PLC9515 is a copolymer with 95% L-lactide and 5% caprolactone, resin IV 1.5 dl/g; and Purasorb PLC9032 is a copolymer of 90% L-lactide and 10% caprolactone, resin IV 3.2 dl/g. Other polymers are envisioned and may be bioabsorbable or non-bioabsorbable.

The extrusion and heat expansion process helps to form large crystalline orientated at certain angles that will provide radial force of the stent 10 formed from this type of tubing. The heat process also provides a shape memory that ensures the deployed stents 10 will maintain the nasal passage open and stay tightly against the inner peripheral tissue walls of the frontal sinus ostium (FSO) or Ethmoid Sinus(ES) with minimal slippage which is important for accurate delivery of a controlled drug coated or infused within the stent 10. The stent 10 is manufactured to reach the stent's 10 anticipated radial force when the stent 10 reaches normal body temperatures or about 37° C. If the stent 10, for one reason or another, does not reach the anticipated radial force when deployed, the stent 10 is manufactured with acceptable manufacturing tolerance rates wherein the radial force range of the stent 10 still provides enough outward force to ensure the stents 10 keep the nasal passage open while also staying tightly against the inner peripheral tissue walls of the frontal ostium with minimal slippage to ensure drug delivery over the anticipated time period.

Once the tubular shape of the polymer stent 10 is formed, the polymer stent 10 is then laser cut without damaging the properties of the polymer 10 and without causing a heat effect at the edges of the stent 10. If the edges of the polymer stent 10 are damaged or the struts 15a, 15b or W-shaped members 20a, 20b the polymer stent 10 will not crimp or expand properly and the mechanical properties will be weak and unreliable.

As mentioned above, stent 10 is compressible to a diameter (e.g., about 3 mm OD) suitable for insertion in and delivery from a handheld delivery cannula or inserter, e.g., balloon catheter 100, sized and shaped for insertion into the desired sinus or nasal cavity or passage. In other embodiments, other stents are supplied in a compressed form over the balloon 50 or other radially expandable device having a guide wire or other suitable inserter sized and shaped for insertion into the desired sinus or nasal cavity or passage. Stent 10 is sized so that stent 10 expands to maintain intimate contact with nearby mucosal tissue, or is sized so as to be slightly undersized in the intended treatment area and not maintain such intimate contact.

To fully evaluate the effectiveness of a stent 10 designed to be delivered to the frontal ostium and not only maintain the nasal passage open for several months but also deliver a controlled amount of drug accurately over that same period without slipping, the stent 10 initially needs to be able to survive the rigors of deployment. A surgically naive frontal sinus ostium is commonly about 3 mm in diameter or less, and requires a 70-80 degree curve to be safely accessed. Encountering bone spurs, polyps, or underdeveloped anatomy is not uncommon when trying to access this area.

During a balloon sinuplasty procedure, the balloon 50 is inflated to 12 atm of pressure (˜176 psi), allowing the balloon 50 to deform and dilate at least the frontal ostium. Stent 10 while crimped to an outer profile of about 3 mm atop a balloon 50 is disposed on the shaft 112 of the catheter 100 must be able to pass around a bend in the range of about 70 to 80 degrees and subsequently expanded at 12 atm against anatomical resistance, all without losing mechanical function. It is important to note that the initial inflation pressure from the balloon 50 forces the stent 10 against the inner peripheral tissue walls of the frontal ostium and positions the stent 10 at the surgeon's desired location, but the radial force of the stent's 10 shape memory maintains the stent 10 in position thereafter. The balloon 50 may be made from nylon, polyurethane, polyester/PET, thermoplastic elastomers such as commonly sold under the trademark Pebax®, e.g., TPU/PEBAX® and PEBAX®/nylon, high-performance transparent polyamides with high resistance to UV degradation commonly sold under the trademark GRILAMID®, and/or polyamides that are highly elastic with excellent low temperature impact strength commonly sold under the trademark VESTAMID®.

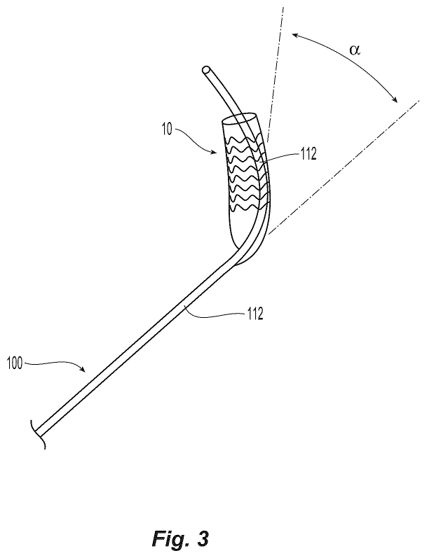

The W-shaped pattern of stent 10 allows stent 10 to be repeatedly crimped and bent atop the balloon catheter 100 (which may in some instances be angled repeatedly upwards to an angle α (up to about 80 degrees) to negotiate the various nasal pathways to gain access to the frontal sinus or other nasal passageways) all without a noticeable loss of integrity (FIG. 3). Stent 10 is also capable of generating a radial force of about 3-5 times other known prior art stents (e.g., the PROPEL™ and the SINUVA™) while maintaining the radial force over very long periods of time, e.g., up to and exceeding three (3) months and in some cases up to and exceeding six (6) months.

Stent 10 is also capable of consistently eluting (delivering) one or more drugs over a period of up to six (6) months (e.g., about 2700 mcg of steroid over a prescribed time period) while maintaining an acceptable radial strength over the same period of time. Stent 10 is also configured to maintain substantially (within 5%) the same length “L” (See FIGS. 1A and 2A) from a crimped configuration to an expanded configuration due to the design of the W-shaped pattern.

Stent 10 may be flexible, bendable, malleable or resilient when manipulated by hand. In some embodiments the stent 10 will not be flexible or bendable unless subjected to greater forces than may be applied by hand, such as the forces imparted by the balloon 50 or other expansion device or tool. As mentioned above, the stent 10 may be coated with an appropriate drug-eluting polymer to deliver a drug to the intended treatment site. Exemplary non-biodegradable polymers that may be used to provide a drug-eluting coating on stent 10 include acrylonitrile butadiene styrene (ABS), polyacrylates and polymethacrylates (e.g., polymethyl methacrylate or polybutyl methacrylate), nylon, polyolefins (e.g., polyethylene or polypropylene), phosphorylcholine (PC), polystyrene, polycarbonate, non-degradable polyesters, polysulfones, polyethersulfones, polyether block amides (e.g., PEBAX™ from Arkema), thermoplastic elastomers (e.g. C-Flex™ from Saint-Gobain Performance Plastics), fluorinated polymers (e.g. polytetrafluoroethylene) or silicones.

Exemplary biodegradable polymers that may be used to provide a drug-eluting coating thereon include synthetic polymers such as polylactic acid (PLLA), poly(lactic-co-glycolic acid) (PLGA), polyglycolic acid, polycaprolactones (PCL) such as poly-E-caprolactone, degradable polyesters (e.g., polyhydroxypropionate, polyhydroxybutyrate and polyhydroxyvalerate), polyanhydrides, polyorthoesters, degradable polycarbonates, degradable polyamides, polyphosphoesters, polyphosphazenes and polycyanoacrylates, and natural polymers such as polysaccharides, proteins and nucleic acids. Exemplary polysaccharides include agars, alginates, carrageenans, celluloses, chitins, chitosans, chondroitin sulfates, dextrans, galactomannans, glycogens, hyaluronic acids, starches, derivatives (including oxidized polysaccharides and salts) of any of the foregoing, and mixtures of any of the foregoing.

In drug-eluting embodiments of the disclosed stent 10, the stent 10 may be impregnated, dip coated, spray coated, or conjugated with a medicament or other therapeutic agent. For example, biodegradable or non-biodegradable polymers like those discussed above may be blended with a suitable drug to provide a drug-eluting polymeric coating on the stent 10. Exemplary therapeutic agents include angiotensin converting enzyme (ACE) inhibitors; angiotensin receptor blockers (ARBS); antihistamines; corticosteroids (e.g., fluticasones such as fluticasone propionate, mometasones such as mometasone furoate, beclomethasone, triamcinolone, flunisolide, budesonide and ciclesonide); non-steroidal anti-inflammatory agents; chymase inhibitors; cyclooxygenase-2 (COX-2) inhibitors; decongestants (e.g., ephedrine, levomethamphetamine, naphazoline, oxymetazoline, phenylephrine, phenylpropanolamine, propylhexedrine, synephrine, tetrahydrozoline, xylometazoline, pseudoephedrine and tramazoline); matrix metalloproteinase (MMP) inhibitors (e.g., doxycycline, TIMP metallopeptidase inhibitor 1 and dexamethasone); mucolytics; opioids (e.g., methadone, morphine, tramadol and oxycodone); therapeutic polymers and combinations thereof. The stent 10 may also include colorants, radiopaque or radiographic fillers or other additives in the stent 10 or in a coating on the stent 10 to aid in visualization or navigation depending upon a particular purpose.

If desired, other therapeutic agents for the treatment or prevention of various conditions may be employed, including analgesics, anti-cholinergics, anti-fungal agents, anti-parasitic agents, antiviral agents, biostatic compositions, chemotherapeutic/antineoplastic agents, cilia enhancement agents (e.g., zinc or magnesium), cytokines, hemostatic agents (e.g., thrombin), immunosuppressors, nucleic acids, peptides, proteins, vasoconstrictors, vitamins, mixtures thereof, and additional other therapeutic agents that will be familiar to persons having ordinary skill in the art. A useful list of such other therapeutic agents may be found, for example, in U.S. Patent Application Publication No. US 2007/0264310 A1 (Hissong et al.), the disclosure of which is incorporated herein by reference.

The stent 10 may include a coating in the form of a liquid, gel or a soluble or insoluble solid. The coating may for example be a drug-eluting coating to enhance healing, an antimicrobial coating to resist formation of biofilms or other infection markers, a lubricating coating to enhance installation, a hemostatic coating to control bleeding, an adhesive coating to enhance retention, or a cilia growth-promoting coating to enhance reciliation. The coating may for example be on an outer or inner surface of the stent 10, or any combination thereof. The coating may be inorganic or organic, and, if organic, may be un-crosslinked, crosslinkable or crosslinked.

The stent 10 may have a variety of diameters or average diameters measured perpendicular to the stent's 10 longitudinal central axis. The stent 10 diameter may, for example, vary dependent upon the intended installation site and patient. A stent 10 for use in an adult human ethmoid or maxillary sinus may for example have an uncompressed diameter (e.g., an average diameter for a stent 10 having a circular or substantially circular cross-section, or a minimum or maximum diameter for a stent 10 having an oval or other noncircular cross-section) less than about 6 cm, than about 5 cm, less than about 4 cm, less than about 3 cm or less than about 2 cm. Such stents 10 may for example have a radially compressed diameter (e.g., an average diameter for a stent having a circular or substantially circular cross-section, or a minimum or maximum diameter for a stent 10 having an oval or other non-circular cross-section) of less than about 1 cm, less than about 9 mm, less than about 8 mm, less than about 7 mm, less than about 6 mm, less than about 5 mm, less than about 4 mm or less than about 3 mm. Expressed using outside diameters from the French catheter scale, these compressed diameters correspond approximately to French Gauge sizes of less than about 30, less than about 26 to 28, less than about 24, less than about 20 to 22, less than about 18, less than about 15, less than about 12 or less than about 10. The same diameters may be used to describe the inside diameter of the hollow tubular portion in the recited inserter.

Stent 10 may have a variety of lengths as measured along the stent's 10 longitudinal central axis. The stent's 10 length may, for example, vary dependent upon the intended installation site. By way of example, the original version of the PROPEL™ stent has a nominal expanded length (which in a typical installation site may correspond to the installed length) of about 23 mm. The PROPEL Mini™ stent has a nominal expanded length of about 16 mm. A stent for use in the ethmoid sinus may for example have an installed length of about 0.1 cm to about 4 cm, e.g., about 1 cm to about 3 cm or about 1.5 cm to about 2 cm.

Stents for use in the frontal or maxillary sinus cavities or the openings thereto may have a variety of lengths, with stents placed in the opening to a frontal or maxillary sinus typically having a shorter length than stents placed in the sinus cavity itself. Frontal or maxillary sinus stents may for example have an installed length of about 0.1 cm to about 5 cm, e.g., about 1 cm to about 4 cm or about 2 cm to about 3 cm. Stents for use in a nasal passage may for example have an installed length of about 0.5 cm to about 6 cm, e.g., about 1 cm to about 5 cm or about 1 cm to about 4 cm. The stent length prior to installation typically will be longer than the installed length and may become shorter when the stent radially expands or is radially expanded during installation.

Not only is it envisioned that stent 10 comes in a variety of lengths to fit a variety of surgical purposes but stent 10 is also designed to remain consistent in length both prior to installation, during expansion, after delivery and over the prescribed treatment period. In other words and as mentioned above, the W-shaped pattern of stent 10 provides a consistent length “L” when disposed in the compressed condition atop the shaft 112 and balloon 50 and after the stent 10 is expanded and delivered within the frontal ostium (or other nasal passage e.g., ethmoid sinus), the length “L” remains essentially the same.

Stent 10 may be packaged and shipped in a radially compressed state, e.g. inside an inserter over the balloon 50 of a catheter 100 or atop a shaft 112 for selective engagement with a catheter 100. The stent 10 and shaft 112 may be assembled with the catheter 100 prior to shipment or prior to surgery the catheter 100 is assembled with the shaft 112 and stent 10 which is separately packaged and sterilized. Variously-sized stents 10 may be shipped with the catheter 100 offering the surgeon sizing options for different patient types.

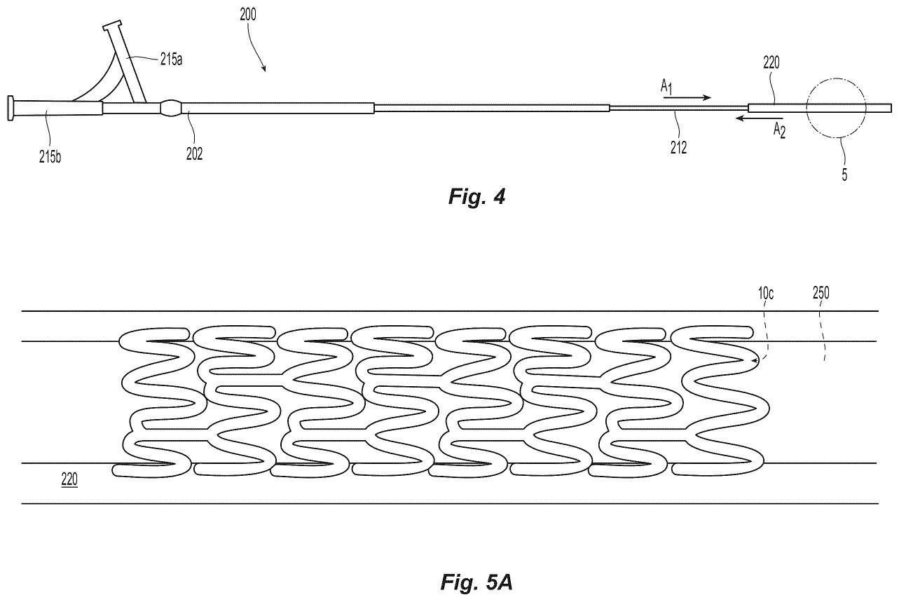

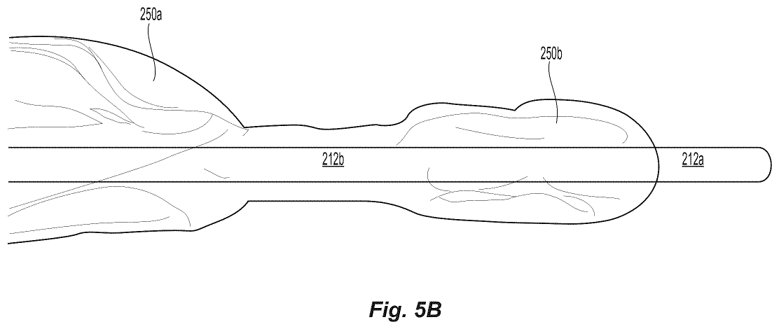

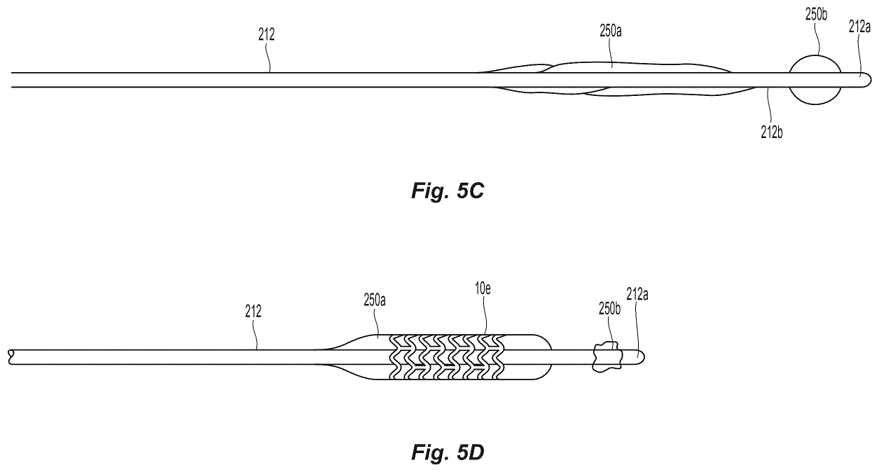

FIGS. 1A-FIG. 3 show one envisioned catheter 100 for use with the presently disclosed stent 10 and balloon 50 disclosed herein. Other balloon catheter styles are envisioned for reliably delivering, positioning and expanding the stent 10. For example, FIGS. 4-5D shows another envisioned catheter 200 configured to work with a dual balloon access and delivery system 250. Catheter 200 includes a proximal end 202 configured to support dual inflation ports 215a and 215b the purposes of which being explained in further detail below. Shaft 212 extends distally from proximal end 202 and supports sleeve 220 thereon. In embodiments, sleeve 220 contains the balloon 50 and stent 10 during handling and is removed prior to use in a patient. For example, it is envisioned that sleeve 220 may be selectively retractable in the direction A2 to expose the dual balloon access and delivery system 250 or held stationary while shaft 212 is selectively extended distally in the direction A1 to expose the dual balloon access and delivery system 250.

Stent 10 is shown crimped and loaded atop shaft 212 and within sleeve 220 in the enlarged transparent detailed view of FIG. 5A. The dual balloon access and delivery system 250 consists of two independently inflatable balloons, delivery balloon 250a and access balloon 250b. Each balloon 250a, 250b connects to a separate inflation port 215a, 215b, e.g., inflation port 215a connects to delivery balloon 250a and inflation port 215b connects to access balloon 250b (FIG. 5B). During surgical navigation to the frontal ostium (or through another nasal passage), the surgeon may encounter various obstructions such as polyps, mucus, abnormal, infected or swollen tissue, small bone spurs which may need to be safely dissected in order for the surgeon to properly deliver the delivery balloon 250a to a desired location. In this instance, the surgeon can inflate the access balloon 250b by opening inflation port 215b while keeping inflation port 215a closed (and stent 10 crimped).

Access balloon 250b can be repeatedly inflated for this purpose as many times as needed to clear the surgical site for stent 10 delivery (see FIG. 5C-stent 10 not shown in this figure for clarity). It is envisioned that access balloon 250b may be made from the same or a different material than delivery balloon 250a due to the nature of its purpose, e.g., access balloon 250b may need to be made from a more durable material due to dissection. Access balloon 250a is disposed proximate a tip 212a of shaft 212 which may also be configured to aide in dissection and is separated by a gap 212b which serves as a common point of attachment between both balloons 250a and 250b.

In order to deliver a stent using catheter 200 and the dual balloon access and delivery system 250, shaft 212 with a dual balloon access and delivery system 250 is loaded with stent 10 onto the catheter 200. The inflation ports 215a, 215b are initially set to zero (or negative) atmospheric pressure to collapse the balloons 250a, 250b onto the shaft 212 to reduce the overall profile to as small as possible for access and delivery purposes. The loading of the stent 10 may be performed as part of manufacturing assembly or by the surgeon in use. The surgeon then begins to navigate the intended nasal passage towards the target area, e.g., frontal sinus ostium.

If the surgeon encounters any obstructions along the delivery path or which the surgeon thinks may cause an issue with proper delivery and placement of the stent 10, the surgeon positions the access balloon 250b and opens inflation port 215b and inflates access balloon 250b to bluntly remove the obstruction (FIG. 5C). Once all of the obstructions are cleared, the surgeon closes inflation port 215b and opens inflation port 215a to expand balloon 250a and deliver the stent 10 against the inner peripheral tissue walls of the frontal ostium as explained above (FIG. 5D).

The radial force of the stent 10 maintains the stent 10 in place against the inner peripheral tissue walls while reliably maintaining the nasal passage open and consistently and continually eluting drugs over a prolonged period of time lasting upwards of three (3) to six (6) months or longer. Once the stent 10 is safely delivered, balloon 250a is deflated and the catheter 200 is removed.

A catheter for delivering a nasal stent includes a housing including an elongated shaft extending distally therefrom; a pair of inflation ports disposed on the housing, each inflation port communicating with a lumen defined through the elongated shaft; an access balloon disposed proximate a distal end of the shaft, the access balloon communicating with a first inflation port of the pair of inflation ports; a delivery balloon disposed proximal to the access balloon and communicating with a second inflation port of the pair of inflation ports, the delivery balloon, when selectively inflated, expanding the delivery balloon; a nasal stent crimped atop the delivery balloon, the nasal stent being made from a material allowing the nasal stent to: expand against the inner peripheral tissue walls of a nasal cavity upon inflation of the delivery balloon; maintain radial strength after expansion; and elute a medicament or therapeutic agent to the inner peripheral tissue walls of the nasal cavity; a balloon actuator configured to selectively and independently regulate the inflation of each balloon; and a nasal stent delivery actuator configured to regulate the exposure of the nasal stent from the sleeve

The catheter for delivering a nasal stent may be configured wherein the access balloon and the delivery balloon are made from different materials

The catheter for delivering a nasal stent may be configured wherein the nasal stent includes an elongated tubular body having proximal and distal ends extending along a longitudinal axis, the elongated body including a repeating pattern of W-shaped members extending around the tubular body separated by longitudinally-oriented and offset struts.

The catheter for delivering a nasal stent may be configured wherein the pattern of the W-shaped members allows the nasal stent to maintain substantially the same radial strength for at least three months.

The catheter for delivering a nasal stent may be configured wherein the pattern of the W-shaped members allows the nasal stent to maintain substantially the same radial strength for at least six months.

The catheter for delivering a nasal stent may be configured wherein the nasal stent is made from a material that consistently elutes a medicament or therapeutic agent into tissue while the pattern of the W-shaped members of the nasal stent maintains substantially the same radial strength against the tissue for at least three months.

The catheter for delivering a nasal stent may be configured wherein the nasal stent is made from a material that consistently elutes a medicament or therapeutic agent into tissue while the pattern of the W-shaped members of the nasal stent maintains substantially the same radial strength against the tissue for at least six months.

The catheter for delivering a nasal stent may be configured wherein the nasal stent is about 3 mm when crimped and about 7 mm when expanded after delivery.

The catheter for delivering a nasal stent may be configured wherein the nasal stent is made from at least one of the following materials Purasorb PLG1017, Purasorb PLG8531, Purasorb PLC9032, Purasorb PL32 and Purasorb PLC9515.

The catheter for delivering a nasal stent may be configured wherein the nasal stent is at least one of impregnated, dip coated, spray coated, or conjugated with a medicament or therapeutic agent.

The catheter for delivering a nasal stent may be configured wherein a biodegradable or non-biodegradable polymer is blended with the medicament or therapeutic agent to provide a drug-eluting polymeric nasal stent.

The catheter for delivering a nasal stent may be configured wherein the medicament or therapeutic agent is selected from the group consisting of angiotensin converting enzyme (ACE) inhibitors, angiotensin receptor blockers (ARBS), antihistamines, corticosteroids, non-steroidal anti-inflammatory agents, chymase inhibitors, cyclooxygenase-2 (COX-2) inhibitors, decongestants, matrix metalloproteinase (MMP) inhibitors, mucolytics, opioids, analgesics, anti-cholinergics, anti-fungal agents, anti-parasitic agents, antiviral agents, biostatic compositions, chemotherapeutic/antineoplastic agents, cilia enhancement agents, cytokines, hemostatic agents, immunosuppressors, nucleic acids, peptides, proteins, vasoconstrictors, and vitamins.

The catheter for delivering a nasal stent may be configured wherein the nasal stent is coated with an ani-inflammation coating configured to reduce tissue swelling and inflammation, an antimicrobial coating configured to resist the formation of biofilms or other infection markers, a lubricating coating configured to enhance installation, a hemostatic coating configured to control bleeding, an adhesive coating configured to enhance retention, or a cilia growth-promoting coating configured to enhance reciliation.

The catheter for delivering a nasal stent may be configured wherein the excipient used to control the release of the medicament or therapeutic agent is a non-biodegradable polymer selected from the group consisting of acrylonitrile butadiene styrene (ABS), polyacrylates and polymethacrylates, nylon, polyolefins, phosphorylcholine (PC), polystyrene, polycarbonate, non-degradable polyesters, polysulfones, polyethersulfones, polyether block amides, thermoplastic elastomers, fluorinated polymers or silicones.

The catheter for delivering a nasal stent may be configured wherein the excipient used to control the release of the medicament or therapeutic agent is a biodegradable polymer selected from the group consisting of polylactic acid (PLLA), poly(lactic-co-glycolic acid) (PLGA), polyglycolic acid, polycaprolactones (PCL) such as poly-E-caprolactone, degradable polyesters, polyanhydrides, polyorthoesters, degradable polycarbonates, degradable polyamides, polyphosphoesters, polyphosphazenes and polycyanoacrylates, and natural polymers such as polysaccharides, proteins and nucleic acids.

The catheter for delivering a nasal stent may further include: a sleeve for housing the nasal stent atop the delivery balloon during navigation to the surgical site; and a nasal stent delivery actuator configured to regulate the exposure of the nasal stent from the sleeve.

A method of delivering a nasal stent includes: engaging a distal end of an elongated shaft of a catheter with an entry of a nasal passage; inflating an access balloon via a first inflation port of a pair of inflation ports disposed on a housing of the catheter to bluntly dissect obstructions as the elongated shaft is navigated through the nasal passage to an infected area; positioning a delivery balloon disposed atop the elongated shaft in the infected area, the delivery balloon configured to support a nasal stent thereon in a crimped configuration; and inflating the delivery balloon via a second inflation port of the pair of inflation ports disposed on the housing to radially expand the nasal stent against the inner peripheral tissue walls of the nasal passage in the infected area, the nasal stent being made from a material allowing the nasal stent to: expand against the inner peripheral tissue walls of a nasal passage upon inflation of the delivery balloon; maintain radial strength after expansion; and elute a medicament or therapeutic agent to the inner peripheral tissue walls of the nasal passage to treat the infected area.

The method of delivering a nasal stent may be configured wherein the access balloon and the delivery balloon are made from different materials.

The method of delivering a nasal stent may be configured wherein the nasal stent maintains substantially the same radial strength for at least three months and the elutes the medicament or therapeutic agent to the inner peripheral tissue walls of the nasal passage to treat the infected area for at least three months.

The method of delivering a nasal stent may be configured wherein the nasal stent maintains substantially the same radial strength for at least three months and the elutes the medicament or therapeutic agent to the inner peripheral tissue walls of the nasal passage to treat the infected area for at least six months.

A nasal stent includes an elongated tubular body having proximal and distal ends extending along a longitudinal axis, the elongated body including a repeating pattern of W-shaped members extending around the tubular body separated by longitudinally-oriented and offset struts, the elongated tubular body selectively transitionable between an expanded configuration and a crimped configuration, wherein the pattern of W-shaped members and struts interleave allowing the elongated tubular body to maintain substantially the same length when disposed in the expanded configuration and the crimped configuration.

The nasal stent may be configured wherein the pattern of the W-shaped members allows the nasal stent to maintain substantially the same radial strength for at least three months.

The nasal stent may be configured wherein the pattern of the W-shaped members allows the nasal stent to maintain substantially the same radial strength for at least six months.

The nasal stent may be configured wherein the nasal stent is made from a material that consistently elutes a medicament or therapeutic agent into tissue while the pattern of the W-shaped members of the nasal stent maintains substantially the same radial strength against the tissue for at least three months.

The nasal stent may be configured wherein the nasal stent is about 3 mm in the crimped configuration and about 7 mm in the expanded configuration after delivery.

The nasal stent may be configured wherein the nasal stent is made from at least one of the following materials Purasorb PLG1017, Purasorb PLG8531, Purasorb PLC9032, Purasorb PL32 and Purasorb PLC9515.

The nasal stent may be configured wherein the nasal stent is at least one of impregnated, dip coated, spray coated, or conjugated with a medicament or therapeutic agent.

The nasal stent may be configured wherein a biodegradable or non-biodegradable polymer is blended with the medicament or therapeutic agent to provide a drug-eluting polymeric nasal stent.

The nasal stent may be configured wherein the medicament or therapeutic agent is selected from the group consisting of angiotensin converting enzyme (ACE) inhibitors, angiotensin receptor blockers (ARBS), antihistamines, corticosteroids, non-steroidal anti-inflammatory agents, chymase inhibitors, cyclooxygenase-2 (COX-2) inhibitors, decongestants, matrix metalloproteinase (MMP) inhibitors, mucolytics, opioids, analgesics, anti-cholinergics, anti-fungal agents, anti-parasitic agents, antiviral agents, biostatic compositions, chemotherapeutic/antineoplastic agents, cilia enhancement agents, cytokines, hemostatic agents, immunosuppressors, nucleic acids, peptides, proteins, vasoconstrictors, and vitamins.

The nasal stent may be configured wherein the nasal stent is coated with an antimicrobial coating configured to resist the formation of biofilms or other infection markers, a lubricating coating configured to enhance installation, a hemostatic coating configured to control bleeding, an adhesive coating configured to enhance retention, an anti-inflammatory coating configured to reduce tissue swelling and inflammation, or a cilia growth-promoting coating configured to enhance reciliation.

The nasal stent may be configured wherein the excipient that controls the release of the medicament or therapeutic agent is a non-biodegradable polymer selected from the group consisting of acrylonitrile butadiene styrene (ABS), polyacrylates and polymethacrylates, nylon, polyolefins, phosphorylcholine (PC), polystyrene, polycarbonate, non-degradable polyesters, polysulfones, polyethersulfones, polyether block amides, thermoplastic elastomers, fluorinated polymers or silicones.

The nasal stent may be configured wherein the excipient that controls the release of the medicament or therapeutic agent is a biodegradable polymer selected from the group consisting of polylactic acid (PLLA), poly(lactic-co-glycolic acid) (PLGA), polyglycolic acid, polycaprolactones (PCL) such as poly-E-caprolactone, degradable polyesters, polyanhydrides, polyorthoesters, degradable polycarbonates, degradable polyamides, polyphosphoesters, polyphosphazenes and polycyanoacrylates, and natural polymers such as polysaccharides, proteins and nucleic acids.

Although specific and in some cases preferred embodiments have been illustrated and described, it will be appreciated by those of ordinary skill in the art that a variety of alternate or equivalent embodiments calculated to achieve the same purposes may be substituted for the specific embodiments shown and described above. This application is intended to cover any such adaptations or variations of the embodiments discussed herein. Therefore, it is manifestly intended that this invention be limited only by the claims and the equivalents thereof.

What is claimed is:

1. A catheter for delivering a nasal stent, comprising:
a housing including an elongated shaft extending distally therefrom;
a pair of inflation ports disposed on the housing, each inflation port communicating with a lumen defined through the elongated shaft;
an access balloon disposed proximate a distal end of the shaft, the access balloon communicating with a first inflation port of the pair of inflation ports;
a delivery balloon disposed proximal to the access balloon and communicating with a second inflation port of the pair of inflation ports, the delivery balloon, when selectively inflated, expanding the delivery balloon;
a nasal stent crimped atop the delivery balloon, the nasal stent being made from a material allowing the nasal stent to:
expand against the inner peripheral tissue walls of a nasal cavity upon inflation of the delivery balloon;
maintain radial strength after expansion; and
elute a medicament or therapeutic agent to the inner peripheral tissue walls of the nasal cavity;
a balloon actuator configured to selectively and independently regulate the inflation of each balloon; and
a nasal stent delivery actuator configured to regulate the exposure of the nasal stent from the sleeve.

2. The catheter for delivering a nasal stent according to claim 1, wherein the access balloon and the delivery balloon are made from different materials.

3. The catheter for delivering a nasal stent according to claim 1, wherein the nasal stent includes an elongated tubular body having proximal and distal ends extending along a longitudinal axis, the elongated body including a repeating pattern of W-shaped members extending around the tubular body separated by longitudinally-oriented and offset struts.

4. The catheter for delivering a nasal stent according to claim 3, wherein the pattern of the W-shaped members allows the nasal stent to maintain substantially the same radial strength for at least three months.

5. The catheter for delivering a nasal stent according to claim 3, wherein the pattern of the W-shaped members allows the nasal stent to maintain substantially the same radial strength for at least six months.

6. The catheter for delivering a nasal stent according to claim 3, wherein the nasal stent is made from a material that consistently elutes a medicament or therapeutic agent into tissue while the pattern of the W-shaped members of the nasal stent maintains substantially the same radial strength against the tissue for at least three months.

7. The catheter for delivering a nasal stent according to claim 3, wherein the nasal stent is made from a material that consistently elutes a medicament or therapeutic agent into tissue while the pattern of the W-shaped members of the nasal stent maintains substantially the same radial strength against the tissue for at least six months.

8. The catheter for delivering a nasal stent according to claim 1, wherein the nasal stent is about 3 mm when crimped and about 7 mm when expanded after delivery.

9. The catheter for delivering a nasal stent according to claim 1, wherein the nasal stent is made from at least one of the following materials Purasorb PLG1017, Purasorb PLG8531, Purasorb PLC9032, Purasorb PL32 and Purasorb PLC9515.

10. The catheter for delivering a nasal stent according to claim 1, wherein the nasal stent is at least one of impregnated, dip coated, spray coated, or conjugated with a medicament or therapeutic agent.

11. The catheter for delivering a nasal stent according to claim 1, wherein a biodegradable or non-biodegradable polymer is blended with the medicament or therapeutic agent to provide a drug-eluting polymeric nasal stent.

12. The catheter for delivering a nasal stent according to claim 1, wherein the medicament or therapeutic agent is selected from the group consisting of angiotensin converting enzyme (ACE) inhibitors, angiotensin receptor blockers (ARBS), antihistamines, corticosteroids, non-steroidal anti-inflammatory agents, chymase inhibitors, cyclooxygenase-2 (COX-2) inhibitors, decongestants, matrix metalloproteinase (MMP) inhibitors, mucolytics, opioids, analgesics, anti-cholinergics, anti-fungal agents, anti-parasitic agents, antiviral agents, biostatic compositions, chemotherapeutic/antineoplastic agents, cilia enhancement agents, cytokines, hemostatic agents, immunosuppressors, nucleic acids, peptides, proteins, vasoconstrictors, and vitamins.

13. The catheter for delivering a nasal stent according to claim 1, wherein the nasal stent is coated with an ani-inflammation coating configured to reduce tissue swelling and inflammation, an antimicrobial coating configured to resist the formation of biofilms or other infection markers, a lubricating coating configured to enhance installation, a hemostatic coating configured to control bleeding, an adhesive coating configured to enhance retention, or a cilia growth-promoting coating configured to enhance reciliation.

14. The catheter for delivering a nasal stent according to claim 1, wherein the excipient used to control the release of the medicament or therapeutic agent is a non-biodegradable polymer selected from the group consisting of acrylonitrile butadiene styrene (ABS), polyacrylates and polymethacrylates, nylon, polyolefins, phosphorylcholine (PC), polystyrene, polycarbonate, non-degradable polyesters, polysulfones, polyethersulfones, polyether block amides, thermoplastic elastomers, fluorinated polymers or silicones.

15. The catheter for delivering a nasal stent according to claim 1, wherein the excipient used to control the release of the medicament or therapeutic agent is a biodegradable polymer selected from the group consisting of polylactic acid (PLLA), poly(lactic-co-glycolic acid) (PLGA), polyglycolic acid, polycaprolactones (PCL) such as poly-E-caprolactone, degradable polyesters, polyanhydrides, polyorthoesters, degradable polycarbonates, degradable polyamides, polyphosphoesters, polyphosphazenes and polycyanoacrylates, and natural polymers such as polysaccharides, proteins and nucleic acids.

16. The catheter for delivering a nasal stent according to claim 1, further comprising:
a sleeve for housing the nasal stent atop the delivery balloon during navigation to the surgical site; and
a nasal stent delivery actuator configured to regulate the exposure of the nasal stent from the sleeve.

17. A method of delivering a nasal stent, comprising:
engaging a distal end of an elongated shaft of a catheter with an entry of a nasal passage;
inflating an access balloon via a first inflation port of a pair of inflation ports disposed on a housing of the catheter to bluntly dissect obstructions as the elongated shaft is navigated through the nasal passage to an infected area;
positioning a delivery balloon disposed atop the elongated shaft in the infected area, the delivery balloon configured to support a nasal stent thereon in a crimped configuration; and
inflating the delivery balloon via a second inflation port of the pair of inflation ports disposed on the housing to radially expand the nasal stent against the inner peripheral tissue walls of the nasal passage in the infected area, the nasal stent being made from a material allowing the nasal stent to:
expand against the inner peripheral tissue walls of a nasal passage upon inflation of the delivery balloon;
maintain radial strength after expansion; and
elute a medicament or therapeutic agent to the inner peripheral tissue walls of the nasal passage to treat the infected area.

18. The method of delivering a nasal stent according to claim 17, wherein the access balloon and the delivery balloon are made from different materials.

19. The method of delivering a nasal stent according to claim 17, wherein the nasal stent maintains substantially the same radial strength for at least three months and the elutes the medicament or therapeutic agent to the inner peripheral tissue walls of the nasal passage to treat the infected area for at least three months.

20. The method of delivering a nasal stent according to claim 17, wherein the nasal stent maintains substantially the same radial strength for at least three months and the elutes the medicament or therapeutic agent to the inner peripheral tissue walls of the nasal passage to treat the infected area for at least six months.
